(19) United States
(12) Patent Application Publication
(10) Pub. No.: US 2006/0110737 A1
(43) Pub. Date: May 25, 2006

(54) Human tumor-associated gene ct120 on chromosome 17p 13.3 region and the protein encoded by it

(76) Inventors: Jianren Gu, Shanghai (CN); Dafang Wan, Shanghai (CN); Xianghue He, Shanghai (CN)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 10/536,772

(22) PCT Filed: Oct. 8, 2003
(86) PCT No.: PCT/CN03/00845

§ 371 Date: May 26, 2005

(30) Foreign Application Priority Data
Nov. 27, 2002 (CN) 02150730.9

(51) Int. Cl. C12Q 1/68; G01N 33/574; C07H 21/04; C12P 21/06; C07K 14/82; C07K 16/30

(52) U.S. Cl. 435/6; 435/7.23; 435/69.1; 435/320.1; 435/325; 530/350; 536/23.5; 530/388.8

(57) ABSTRACT

The invention related to a novel human tumor associated polypeptide CT120, and the encoding polynucleotide, and to the method for producing the polypeptide by recombinant technology, also to the method for diagnosing and treating diseases such as carcinoma using the polypeptide. The invention also related to antagonists against the polypeptide and its uses for treatment, aslo to the uses of the polynucleotide coding for the human tumor-associated protein.

### FIELD OF INVENTION

This invention relates to the field of biotechnique. Specifically, the present invention relates to a novel polynucleotide encoding human tumor related gene CT120 in the region of sub-band 3 of band 3 of zone 1 of human chromosome 17 (17p13.3), and the polypeptide encoded by said polynucleotide. The present invention further relates to the use and preparation of said polynucleotide and polypeptide.

### BACKGROUND OF INVENTION

The mortality rate of malignant tumor is just lower than that of cardio-and cerebro-vascular disease in China. It is generally believed that the tumor is a disease involving multiple factors and steps.

The occurrence and development of tumor is a process of evolution of a clone accompanied with a series of change of the genetic material in the cell nucleus, including the change of a sequence such as point mutation, deletion or insertion, aberration of structure such as deletion or rearrangement of a large segment, or gene amplification. More and More evidences show that activation and/or inactivation of different genes and their complex mutual interaction exist at different stages in the evolution process of a clone. As a result, the isolation and identification of tumor related genes may deepen the understanding for the mechanism of the occurrence of tumor and be helpful for the prevention, diagnosis, treatment and prognosis of tumor.

Hepatocellular carcinoma (HCC) is a malignant tumor occurred in Asian with high prevalence. Concerning the molecular mechanism of the occurrence of HCC, the biologists on tumor in many labs have noticed at the beginning of 1990's that there is heterozygous deletion in chromosome 17p13.3 region of HCC patients (Fujimori et al. Cancer Res. 1991, 51:89-93; Boige et al, Cancer Res. 1997, 57:1986-1990; Nagai et al, Oncogene, 1997, 14:2927-2933). At the same time, a lab of Shanghai Cancer Institute has found that there is heterozygous deletion with high frequency in chromosome 17p13.3 region in Chinese liver cancer patient, indicating that there is one or several cancer-inhibiting genes in chromosome 17p13.3 region where the heterozygous deletion with high frequency occurs. These genes may be different from p53 gene and play important role in the occurrence and development of liver cancer. Later, this lab firstly determines that the minimal range of heterozygous deletion in hepatocarcinoma patients is 0.5 Mb (Wang et al, Genes Chromosomes & Cancers, 2001, 31:221-227).

Since cancer is one of the main diseases harmful to human health, people are concerned about the early diagnosis and gene therapy of cancer so as to effectively cure and prevent tumors, such as hepatocarcinoma. Therefore, there is a keen need in the art to develop new cancer-related human proteins and its agonist and inhibitor.

### SUMMARY OF INVENTION

One purpose of the invention is to provide a novel tumor related protein, named CT120 protein, and its fragments, analogs and derivatives.

Another purpose of the invention is to provide polynucleotides encoding said polypeptides.

Still another purpose of the invention is to provide a method for producing said polypeptides and the use of said polypeptide and encoding sequence.

In the first aspect, the invention provides an isolated human CT120 polypeptide, which comprises the amino acid sequence of SEQ ID NO: 2, or its conservative variant polypeptides, active fragments or active derivatives. Said polypeptide preferably is selected from the following group: (a) a polypeptide having the amino acid sequence of SEQ ID NO: 2; (b) a polypeptide derived from the polypeptide (a) with one or more substitution, deletion or insertion in the amino acid residues of polypeptide (a), which has the function of promoting the growth of NIH/3T3 cell.

The second aspect of the present invention provides an isolated polynucleotide, which comprises a nucleic acid sequence having at least 85% sequence identity with a nucleic acid sequence selected from the following group: (a) polynucleotides encoding the polypeptide according to claims 1 and 2; (b) polynucleotides which are complementary to polynucleotides (a). Preferably, the polypeptide encoded by said polynucleotide has the amino acid sequence of SEQ ID NO: 2. More preferably, said polynucleotide has a sequence selected from the following group: the sequence in encoding region (position 91-861) of SEQ ID NO: 1 or the full length of SEQ ID NO: 1.

The third aspect of the present invention provides a vector comprising the above polynucleotide and a host cell transformed or transduced by said vector or a host cell transformed or transdues directly by the above polynucleotide.

In the fourth aspect, the invention provides a method for producing a polypeptide having the activity of tumor-related protein CT 120, which comprises (a) culturing the above transformed or transduced host cell under the conditions suitable for the expression of protein; (b) isolating the polypeptides having the activity of tumor-related protein CT120 from the culture.

In the fifth aspect, the invention provides an antibody specifically binding with the above polypeptides of tumor-related protein CT120. The present invention further provides a nucleic acid molecule useful for detection, which comprises consecutive 20-150 nucleotides in the above polynucleotide.

The sixth aspect of the present invention provides a pharmaceutical composition containing safe and effective amount of antagonists (such as antisense sequence or antibody) of tumor-related protein CT120, as well as pharmaceutically acceptable carrier. Said pharmaceutical composition may be used to treat diseases such as cancer and abnormal proliferation of cells.

The seventh aspect of the present invention provides a method for detecting the carcinomatous change or susceptibility for carcinomatous change in pneumonocytes, comprising the steps of: detecting whether there is any CT120 transcript in the pneumonocyte sample, the presence of CT120 transcript indicating that said pneumonocyte undergoes carcinomatous change or has the susceptibility for carcinomatous change; or detecting whether there is CT120 protein in the pneumonocyte sample, the presence of CT120 target indicating that said pneumonocyte undergoes carcinomatous change or has the susceptibility for carcinomatous change.

The eighth aspect of the present invention provides a kit for detecting pulmonary cancer, which comprises (1) primers which specifically amplify human CT120 gene, or (2) antibody specifically binding with CT120 protein.

The other aspects of the invention will be apparent to the skilled in the art in light of the technical disclosure of the invention.

### DESCRIPTION OF DRAWINGS

FIG. 1 shows the result of sequence alignment between CT120 and four homologous sequences.

FIG. 2 shows the Northern hybridization result of multiple tissues of CT120, wherein each lane represents the following: 1. heart; 2. brain; 3. placenta; 4. lung; 5. liver; 6. skeletal muscle; 7. kidney; 8. pancreas.

FIG. 3 shows the expression of CT120 in different tumor tissue, wherein each lane represents the following: 1. SPC-A-1; 2. C-33A; 3. SMMC-7721; 4.BEL-7402; 5.SK-OV-3; 6. 5637; 7. A431; 8. MCF-7.

FIG. 4 shows the result of NIH/3T3 cell transfected by CT120.

FIG. 5 shows the detection of the expression of CT120 in stably transfected cell line by Western blotting assay, wherein lanes 1-6 represent 6 clones, respectively.

FIG. 6 show the detection of the expression of CT120 in the tissue of pulmonary cancer and the tissue near the pulmonary cancer by immunohistochemistry assay, wherein A represents the tissue of pulmonary cancer and B represents the tissue near the pulmonary cancer.

### DETAILED DESCRIPTION OF INVENTION

In the hepatocarcinoma study, the inventors first found that there was high frequency of LOH (60-100%) of 17p13.3 in hepatocarcinoma (HC) tissue. Recently, the full genomic scanning of HC also proved that 17p13.3 was the region having the highest frequency. The inventors isolated and cloned the tumor related ESTs or expressed sequence tags in region 17p13.3. By using PAC P579 clone corresponding to site 926 in region 17p13.3, the cDNA clone was obtained and then was sequenced via 9 times shotgun method. Among them, an EST representing a novel gene, named CT120, was found by using computer analysis. The full-length nucleotide sequence and the encoded amino acid sequence thereof were obtained by RACE. The Northern blotting and Southern blotting results proved that the expression of CT120 in pulmonary cancer tissue was high but there was substantially no expression in the tissue near pulmonary cancer. That means CT120 was related to tumor. The in vitro experiment proved that CT120 had the function of promoting the transformation of mice NIH/3T3 cell. As a result, CT120 is a candidate gene which may be used in the diagnosis, treatment and prevention of tumor.

As used herein, the terms “CT120 protein”, “CT120 polypeptide”, “tumor related CT120 protein” or “tumor related CT120 protein” are exchangeable, referring to a protein or polypeptide comprising the amino acid sequence of natural human tumor related protein CT120 (SEQ ID NO: 2). The terms include CT120 with or without the starting Met residue.

As used herein, the term “isolated” refers to a substance which has been isolated from the original environment. For naturally occurring substance, the original environment is the natural environment, e.g., the polynucleotide and polypeptide in a naturally occurring state in the viable cells are not isolated or purified. However, if the same polynucleotide and polypeptide have been isolated from other components naturally accompanying them, they are isolated or purified.

As used herein, the terms “isolated tumor related CT120 protein or polypeptide” or “isolated CT120 protein or polypeptide” mean that CT120 polypeptide does not essentially contain other proteins, lipids, carbohydrate or any other substances associated therewith in nature. The artisans can purify CT120 protein by standard protein purification techniques. The substantially pure polypeptide may produce a single main band on non-reduced SDS-PAGE. The purity of CT120 protein or polypeptide may be analyzed via amino acid sequencing.

The polypeptide of invention may be a recombinant, natural, or synthetic polypeptide, preferably a recombinant polypeptide. The polypeptide of invention may be a purified natural product or a chemically synthetic product. Alternatively, it may be produced from prokaryotic or eukaryotic hosts, such as bacteria, yeast, higher plant, insect, and mammalian cells, using recombinant techniques. According to the host used in the recombinant production, the polypeptide may be glycosylated or non-glycosylated. The polypeptide may or may not comprise the starting Met residue.

The invention further comprises the fragments, derivatives and analogues of CT120. As used in the invention, the terms “fragment”, “derivative” and “analogue” mean the polypeptide that essentially retains the same biological functions or activity of natural tumor related CT120 protein. The fragment, derivative or analogue of the present polypeptide may be (i) one in which one or more of the amino acid residues are substituted with a conserved or non-conserved amino acid residue (preferably a conserved amino acid residue), or (ii) one in which one or more of the amino acid residues include a substituent group, or (iii) one in which the mature polypeptide is fused with another compound, such as a compound for increasing the half-life of the polypeptide (for example, polyethylene glycol), or (iv) one in which the additional amino acids are fused to mature polypeptide, such as a leader or secretary sequence or a sequence used for purifying polypeptide or protein. Such fragments, derivatives and analogs are known to the artisans based on the teachings herein.

In the present invention, the terms “human tumor related protein CT120 polypeptide” and “human NIP 2 AP protein polypeptide” are exchangeable. They both mean a polypeptide having the activity of human tumor related CT120 protein comprising the amino acid sequence of SEQ ID NO: 2. The term also comprises the variants of sequence SEQ ID NO: 2 which have the same function of human tumor related protein CT120. These variants include, but are not limited to, deletions, insertions and/or substitutions of several amino acids (typically 1-50, preferably 1-30, more preferably 1-20, most preferably 1-10), and addition of one or more amino acids (typically less than 20, preferably less than 10, more preferably less than 5) at C-terminus and/or N-terminus. For example, the functions of the protein are usually unchanged when an amino residue is substituted by a similar or analogous one. Further, the addition of one or several amino acids at C-terminus and/or N-terminus usually does not change the protein function. The term also includes the active fragments and derivatives of human tumor related protein CT120.

The variants of polypeptide include homologous sequences, conservative variants, allelic variants, natural mutants, induced mutants, proteins encoded by DNA which hybridizes to human tumor related protein CT120 DNA under high or low stringency conditions, as well as the polypeptides retrieved by antisera raised against human tumor related protein CT120 polypeptide. The present invention also provides other polypeptides, e.g., fusion proteins, which include the human tumor related protein CT120 polypeptide or fragments thereof. Besides substantially full-length polypeptide, the soluble fragments of human tumor related protein CT120 polypeptide are also included. Generally, these fragments comprise at least 10, typically at least 30, preferably at least 50, more preferably at least 80, most preferably 100 consecutive amino acids of human tumor related protein CT120 polypeptide.

The invention also provides the analogues of human tumor related protein CT120 polypeptide. Analogues can differ from naturally occurring CT120 polypeptide by amino acid sequence differences or by modifications which do not affect the sequence, or by both. These polypeptides include genetic variants, both natural and induced. Induced variants can be made by various techniques, e.g., by random mutagenesis using irradiation or exposure to mutagens, or by site-directed mutagenesis or other known molecular biologic techniques. Also included are analogues which include residues other than those naturally occurring L-amino acids (e.g., D-amino acids) or non-naturally occurring or synthetic amino acids (e.g., beta- or gamma-amino acids). It is understood that the polypeptides of the invention are not limited to the representative polypeptides listed above.

Modifications (which do not normally alter primary sequence) include in vivo or in vitro chemical derivation of polypeptides, e.g., acetylation, or carboxylation. Also included are modifications of glycosylation, e.g., those made by modifying the glycosylation patterns of a polypeptide during its synthesis and processing or in the further processing steps, e.g., by exposing the polypeptide to glycosylation enzymes (e.g., mammalian glycosylating or deglycosylating enzymes). Also included are sequences having phosphorylated amino acid residues, e.g., phosphotyrosine, phosphoserine, phosphothronine, as well as sequences modified to improve the resistance to proteolytic degradation or to optimize solubility properties.

In the invention, “conservative mutant of human tumor related protein CT120” means a polypeptide formed by substituting at most 10, preferably at most 8, more preferably 5, and most preferably at most 3 amino acids with the amino acids having substantially the same or similar property, as compared with the amino acid sequence of SEQ ID NO: 2. Preferably, these conservative mutants are formed by the substitution according to Table 1.

TABLE 1

| | Representative | Preferred |
|---|---|---|
| Initial residue | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The polynucleotide of invention may be in the forms of DNA and RNA. DNA includes cDNA, genomic DNA, and synthetic DNA, etc., in single strand or double strand form. A single strand DNA may be an encoding strand or non-encoding strand. The coding sequence for mature polypeptide may be identical to the coding sequence shown in SEQ ID NO: 1 (positions 96-861), or is a degenerate sequence. As used herein, the term “degenerate sequence” means an sequence which encodes a protein comprising the sequence of SEQ ID NO: 2 and which has a nucleotide sequence different from the coding region in SEQ ID NO: 1.

The sequences encoding the mature polypeptide of SEQ ID NO: 2 include those encoding only the mature polypeptide, those encoding mature polypeptide plus various additional encoding sequence, the sequence encoding for mature polypeptide plus the non-encoding sequence and optional additional encoding sequence.

The term “polynucleotide encoding the polypeptide” includes the polynucleotide encoding said polypeptide and the polynucleotide comprising additional and/or non-encoding sequence.

The invention further relates to the variants of polynucleotides which encode a polypeptide having the same amino acid sequence, or its fragment, analogue and derivative. The variant of the polynucleotide may be a naturally occurring allelic variant or a non-naturally occurring variant. Such nucleotide variants include substitution, deletion, and insertion variants. As known in the art, the allelic variant is a substitution form of polynucleotide, which may be a substitution, deletion, and insertion of one or more nucleotides without substantially changing the functions of the encoded polypeptide.

The invention further relates to polynucleotides, which hybridize to the hereinabove-described sequences, if there is at least 50%, preferably at least 70%, more preferably at least 80%, and most preferably 90% between the sequences. The invention particularly relates to polynucleotides, which hybridize under stringent conditions to the polynucleotides of the invention. As herein used, the term “stringent conditions” means the following conditions: (1) hybridization and washing under relatively low ionic strength and relatively high temperature, such as 0.2×SSC, 0.1% SDS, 60° C.; (2) hybridization after adding denaturants, such as 50% (v/v) formamide, 0.1% bovine serum/0.1% Ficoll, 42° C.; or (3) hybridization of two sequences sharing at least 95%, preferably 97% homology. Further, the hybridizing polynucleotides encode a polypeptide which retains the same biological function or activity as the mature polypeptide of SEQ ID NO:2.

The invention also relates to nucleic acid fragments hybridized with the hereinabove sequence. As used herein, the length of “nucleic acid fragment” is at least 15 bp, preferably 30 bp, more preferably 50 bp, and most preferably at least 100 bp. These fragments can be used in the amplification techniques of nucleic acid, e.g., PCR, to determine and/or isolate the CT120 encoding polynucleotide.

The polypeptide and polynucleotide of the invention are preferably in isolated form, preferably purified to be homogenous.

According to the invention, the DNA sequence can be obtained in various ways. For example, the DNA is isolated by the hybridization techniques well-known in the art, which includes, but are not limited to, 1) the hybridization between the probe and genomic or cDNA library so as to select the homologous nucleotide sequence, and 2) expression of the antibodies against the library so as to screen out the DNA fragments having the common structure features.

The specific DNA fragment sequences encoding CT120 protein may further be obtained by the following methods so as to obtain the double-stranded DNA for said polypeptide: 1) isolating double-stranded DNA sequence from genomic DNA; and 2) chemical synthesis of DNA sequence.

In the above methods, the isolation of genomic DNA is least frequently used. The direct chemical synthesis of DNA sequence is commonly used when the whole amino acid sequence of the desired polypeptide product is known. When the whole amino acid sequence of the desired polypeptide product is not known, the direct chemical synthesis of DNA sequence is impossible and the available method is to isolate cDNA sequence. The standard method for isolating the cDNA of interest is to isolate mRNA from donor cells that highly express said gene followed by reverse transcription of mRNA to form plasmid or phage cDNA library. There are many sophisticated techniques for extracting mRNA and the kits are commercially available (Qiagene). The conventional method can be used to construct cDNA library. The cDNA libraries are also commercially available, e.g., different cDNA libraries from Clontech. When PCR is used in combination, even an extremely small amount of expression products can be cloned.

The conventional methods can be used for screening the gene of invention from a cDNA library. These methods include, but are not limited to, (1) DNA-DNA or DNA-RNA hybridization, (2) the appearance or loss of the function of marker gene, (3) the determination of the level of CT120 protein transcripts, (4) the determination of protein product of gene by immunology methods or the biological activity assays. These methods can be used alone or in combination.

In method (1), the probe used in the hybridization may be identical to any portion of polynucleotide of the present invention. The length of probe is at least 15, preferably at least 30, more preferably at least 50, and most preferably at least about 100 nucleotides. Additionally, the length of probe is usually less than 2 kb, preferably less than 1 kb. The probe usually is the DNA sequence chemically synthesized on the basis of the sequence information of gene of the present invention. Of course, the gene of the present invention itself or the fragment thereof can be used as a probe. The labels for DNA probe include, e.g., radioactive isotopes, fluoresceins or enzymes, such as alkaline phosphatase.

In method (4), the detection of the protein products expressed by CT120 gene can be carried out by immunology methods, such as Western blotting, radioimmunoassay, ELISA and so on.

The method of amplification of DNA/RNA by PCR is preferably used to obtain the gene of the present invention. Especially when it is difficult to obtain the full-length cDNA, the method of RACE is preferably used. The primers used in PCR can be properly selected according to the sequence information of the present invention disclosed herein and synthesized by conventional methods. The amplified DNA/RNA fragments can be isolated and purified by conventional methods such as gel electrophoresis.

For the gene of the present invention or its DNA fragments, the sequencing of polynucleotide sequence can be carried out by conventional dideoxy sequencing method (Sanger et al. PNAS, 1977, 74: 5463-5467). The sequencing of nucleotide sequence can also be performed using commercially available sequencing kits. In order to obtain the full-length cDNA sequence, it is necessary to repeat the sequencing. Sometimes, it may be necessary to sequence the DNA of several clones to obtain the full-length cDNA sequence.

The invention further relates to a vector comprising the polynucleotide of the present invention, a genetic engineered host cell transformed with the vector of the present invention or directly with the sequence encoding CT120 protein protein, and the method for producing the polypeptide of the present invention by recombinant techniques.

The recombinant CT120 polypeptides can be expressed or produced by the conventional recombinant DNA technology (Science, 1984; 224:1431), using the polynucleotide sequence of the present invention. Generally, it comprises the following steps:

(1) transfecting or transforming the appropriate host cells with the polynucleotide encoding human tumor related CT120 protein of the present invention or the vector containing said polynucleotide;

(2) culturing the host cells in an appropriate medium;

(3) isolating or purifying the protein from the medium or cells.

In the present invention, the polynucleotide sequences encoding human tumor related CT120 protein may be inserted into a recombinant expression vector. The term “expression vector” refers to a bacterial plasmid, bacteriophage, yeast plasmid, plant virus or mammalian cell virus, such as adenovirus, retrovirus or any other vehicles known in the art. Vectors suitable for use in the present invention include, but are not limited to, the T7-based expression vector for expression in bacteria (Rosenberg, et al., Gene, 56:125, 1987), the pMSXND expression vector for expression in mammalian cells (Lee and Nathans, J. Biol. Chem., 263:3521, 1988) and baculovirus-derived vectors for expression in insect cells. On the whole, any plasmids or vectors may be used to construct the recombinant expression vector as long as it can replicate and is stable in the host. One important feature of expression vector is that the expression vector typically contains an origin of replication, a promoter, a marker gene as well as the translation regulatory components.

The methods known by the artisans in the art can be used to construct an expression vector containing the DNA sequence of CT120 protein and appropriate transcription/translation regulatory components. These methods include in vitro recombinant DNA technique, DNA synthesis technique, in vivo recombinant technique and so on (Sambrook, et al. Molecular Cloning, a Laboratory Manual, Cold Spring Harbor Laboratory. New York, 1989). The DNA sequence is efficiently linked to the proper promoter in an expression vector to direct the synthesis of mRNA. The exemplary promoters are lac or trp promoter of E. coli; P_{L} promoter of λ phage; eukaryotic promoter including CMV immediate early promoter, HSV thymidine kinase promoter, early and late SV40 promoter, LTRs of retrovirus and some other known promoters which control the gene expression in the prokaryotic cells, eukaryotic cells or virus. The expression vector may further comprise a ribosome-binding site for initiating the translation, transcription terminator and the like.

Further, the expression vector preferably comprises one or more selective marker genes to provide a phenotype for the selection of the transformed host cells, e.g., the dehydrofolate reductase, neomycin resistance gene and GFP (green flurencent protein) for eukaryotic cells, as well as tetracycline or ampicillin resistance gene for E. coli.

The vector containing said DNA sequence and proper promoter or regulatory elements can be transformed into appropriate host cells to express the protein. The “host cell” includes prokaryote, such as bacteria; primary eukaryote, such as yeast; advanced eukaryotic, such as mammalian cells. The representative examples are bacterial cells, such as E. coli, Streptomyces, Salmonella typhimurium; fungal cells, such as yeast; plant cells; insect cells such as Drosophila S2 or Sf9; animal cells such as CHO, COS or Bowes melanoma, etc.

Transcription of the polynucleotide of the present invention in higher eukaryotes is increased by inserting an enhancer sequence into the vector. Enhancers are cis-acting elements of DNA, usually about from 10 to 300 bp that act on a promoter to increase the gene transcription. Examples include the SV40 enhancer on the late side of the replication origin 100 to 270 bp, the polyoma enhancer on the late side of the replication origin, and adenovirus enhancers.

The skilled in the art knows clearly how to select appropriate vectors, promoters, enhancers and host cells.

Recombinant transformation of host cell with the DNA sequence of the present invention might be carried out by conventional techniques well known to those skilled in the art. Where the host is prokaryotic such as E. coli, the competent cells, which are capable of DNA uptake, can be prepared from cells harvested after exponential growth phase and subsequently treated by the CaCl₂ method using procedures well known in the art. Alternatively, MgCl₂ can be used. The transformation can also be carried out by electroporation, if desired. When the host is an eukaryote, such methods of transfection of DNA as calcium phosphate co-precipitates, conventional mechanical procedures such as micro-injection, electroporation, or liposome-mediated transfection may be used.

The transformants are cultured using conventional methods to express the polypeptides of the present invention. According to the used host cells, the medium for cultivation can be selected from various conventional mediums. The host cells are cultured under a condition suitable for its growth until the host cells grow to an appropriate cell density. Then, the selected promoter is induced by appropriate means (e.g., temperature shift or chemical induction) and cells are cultured for an additional period.

In the above methods, the recombinant polypeptide may be included in the cells, or expressed on the cell membrane, or secreted out of the cell. If desired, the physical, chemical and other properties can be utilized in various isolation methods to isolate and purify the recombinant protein. These methods are well-known to those skilled in the art and include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, and any other liquid chromatography, and the combination thereof.

The present inventors have found out that CT120 gene is not expressed in normal pulmonary tissue, but is expressed in cells of pulmonary cancer. As a result, pulmonary cancer may be detected by detecting CT120 transcript or protein.

Therefore, the recombinant human tumor related CT120 protein or polypeptide have various uses including, but not to be limited to: screening out antibodies, polypeptides or ligands as agonists or antagonists of CT120 protein. For example, antibodies can be used to inhibit the function of CT120 protein. The expressed recombinant CT120 protein can be used to screen polypeptide library to find out therapeutically valuable polypeptide molecules which inhibit or activate the function of CT120 protein.

The invention also provides the method for screening medicaments so as to identify agents which improve CT120 protein (agonists) or repress CT120 protein (antagonists). For example, in the presence of an agent, the mammal cells or the membrane preparation expressing CT120 protein can be incubated with the labeled CT120 protein to determine the ability of the agent to enhance or repress the interaction.

The antagonists of CT120 protein include the screened antibodies, compounds, deletants and analogues. The antagonists of CT120 protein can bind to CT120 protein and eliminate its function, or inhibit the production of CT120, or bind to the active site of said polypeptide so that the polypeptide can not function biologically. The antagonists of CT120 protein can be used in the therapy.

The antagonists of the polypeptide of the present invention (such as antisense sequence and antibody) can be directly used for the treatment of diseases, e.g., various malignant tumors, abnormal cell proliferation and so on, and especially for the treatment of pulmonary cancer and liver cancer.

The polypeptide, and its fragment, derivative, analogue or cells can be used as antigens to produce antibodies. These antibodies may be polyclonal or monoclonal antibodies. The polyclonal antibodies can be prepared by directly injecting the polypeptide into the animals.

According to the invention, the polypeptides or its antagonists may be employed in combination with a suitable pharmaceutical carrier. Such a carrier includes, but is not limited to, water, glucose, ethanol, salt, buffer, glycerol, and combinations thereof. Such compositions comprise a safe and effective amount of the polypeptide or antagonist, as well as a carrier or excipient which does not influence the effect of the drug. These compositions can be used for treatment of disease.

The invention also provides a pharmaceutical package or kit comprising one or more containers filled with one or more of the ingredients of the pharmaceutical compositions of the present invention. Associated with such container (s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

The pharmaceutical compositions may be administered in a convenient manner such as by the topical, intravenous, intraperitoneal, intramuscular, subcutaneous, intranasal or intradermal routes. The CT120 protein is administered in an amount, which is effective for treating and/or prophylaxis of the specific indication. The amount of CT120 protein administrated on patient will depend upon various factors, such as delivery methods, the subject health, and the like, and is within the judgment of the skilled clinician.

Polynucleotides corresponding to CT120 protein also have many therapeutic applications. Gene therapy technology can be used in the therapy of the abnormal cell proliferation, development or metabolism, which is caused by the abnormal expression of CT120 protein.

Also included in the present invention are ribozyme and the oligonucleotides, including antisense RNA and DNA, which inhibit the translation of CT120 mRNA. Ribozyme is an enzyme-like molecule capable of specifically cutting certain RNA. The mechanism is the nucleic acid endo-cleavage after the specific hybridization of ribozyme molecule and the complementary target RNA.

The methods for introducing the polynucleotides into tissues or cells include: directly injecting the polynucleotides into tissue in the body, in vitro introducing the polynucleotides into cells with vectors, such as virus, phage, or plasmid, and then transplanting the cells into the body.

The invention also provides the antibodies against the determinants of CT120 protein. These antibodies include, but are not limited to, polyclonal antibody, monoclonal antibody, chimeric antibody, single-chain antibody, Fab fragment and the fragments produced by Fab expression library.

The antibody against CT120 protein can be used in immunohistochemical method to detect the presence of CT120 protein in the biopsy specimen.

The monoclonal antibody specific to CT120 protein can be labeled by radioactive isotopes, and injected into human body to trace the location and distribution of CT120 protein. This radioactively labeled antibody can be used in the non-wounding diagnostic method for the mapping of the tumor and determination of the metastasis of tumor cells.

The antibody of the present invention is useful for the therapy or the prophylaxis of disorders related to the CT120 protein. The appropriate amount of antibody can be administrated to stimulate or block the production or activity of CT120 protein.

Antibodies can also be designed as an immunotoxin targeting at the particular site in the body. For example, a monoclonal antibody having high affinity to CT120 protein can be covalently bound to bacterial or plant toxins, such as diphtheria toxin, ricin, ormosine. One common method is to challenge the amino group on the antibody with sulfydryl cross-linking a gents, such as SPDP, and bind the toxin onto the antibody by interchanging the disulfide bonds. This hybrid antibody can be used to kill CT120 protein-positive cells.

The polyclonal antibodies can be prepared by immunizing animals, such as rabbit, mouse, and rat, with CT120 protein. Various adjuvants, e.g., Freund's adjuvant, can be used to enhance the immunization.

The techniques for producing CT120 protein monoclonal antibodies include the hybridoma technique (Kohler and Milstein. Nature, 1975, 256:495-497). The chimeric antibody comprising a constant region of human origin and a variable region of non-human origin can be produced using the conventional method in the art (Morrison et al, PNAS, 1985,81:6851). Furthermore, the techniques for producing single-chain antibody (U.S. Pat. No. 4,946,778) are also useful for preparing the single-chain antibody against CT120 protein.

The polypeptide molecule capable of binding to CT120 protein can be obtained by screening out the random polypeptide library consisting of the various combinations of amino acids bound onto the solid matrix. Typically, CT120 protein is labeled in the screening.

The invention further provides diagnostic assays for quantitative and in situ measurement of CT120 protein level. These assays are well known in the art and include FISH assay and radioimmunoassay.

The polynucleotide encoding CT120 protein can be used in the diagnosis and treatment of CT120 protein related diseases (especially lung cancer). The polynucleotide encoding CT120 protein can be used to detect whether CT120 protein is expressed or not, and whether the expression of CT120 protein is normal or abnormal, e.g., in the case of diseases. CT120 DNA sequences can be used in the hybridization with biopsy samples to determine the abnormal expression of CT120 protein. The hybridization methods include Southern blotting, Northern blotting and in situ blotting, etc., which are public-known and sophisticated techniques. The corresponding kits are commercially available. A part of or all of the polynucleotides of the present invention can be used as probe and fixed on a microarray or DNA chip for analysis the differential expression of genes in tissues and for the diagnosis of genes. The CT120 protein specific primers can be used in RNA-polymerase chain reaction and in vitro amplification to detect the transcripts of CT120 protein.

Further, detection of the mutation of CT120 protein gene is useful for the diagnosis of CT120 protein related diseases (especially lung cancer). The mutation forms of CT120 protein include site mutation, translocation, deletion, rearrangement and any other mutations compared with the wild-type CT120 DNA sequence. The conventional methods, such as Southern blotting, DNA sequencing, PCR and in situ blotting, can be used to detect mutation. Moreover, mutation sometimes affects the expression of protein. Therefore, Northern blotting and Western blotting can be used to indirectly determine whether the gene is mutated or not.

The full length CT120 nucleotide sequence or its fragment of the present invention can be prepared by PCR amplification, recombinant method and synthetic method. For PCR amplification, one can obtain said sequences by designing primers based on the nucleotide sequence disclosed in the invention, especially the sequence of ORF, and using cDNA library commercially available or prepared by routine techniques known in the art as a template. When the sequence is relatively long, it is usually necessary to perform two or more PCR amplifications and link the amplified fragments together in the correct order.

Once the sequence is obtained, a great amount of the sequences can be produced by recombinant methods. Usually, said sequence is cloned in a vector which is then transformed into a host cell. Then the sequence is isolated from the amplified host cells using conventional techniques.

Further, the sequence can be produced by synthesis, especially when the fragment is short. Typically, several small fragments are synthesized and linked together to obtain a long sequence.

At present, it is completely feasible to chemically synthesize the DNA sequence encoding the protein of the present invention, or the fragments or derivatives thereof. In addition, mutations can be introduced into the sequence of the protein by chemical synthesis.

The present invention has demonstrated for the first time that CT120 is expressed in different tumor tissues to different extents. It is especially induced and highly expressed in lung cancer. In vitro DNA transfection assay further proves that CT120 clone has significant effect of promoting the growth of NIH/3T3 cells. As a result, CT120 is a new tumor related gene which has potential application in the diagnosis, treatment and prevention of tumor.

The invention is further illustrated by the following examples. These examples are only intended to illustrate the invention, but not to limit the scope of the present invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

### EXAMPLE 1

### Computerized Prediction of New Gene in PAC 579 Clone

The DNA sequence of PAC 579(P579) clone (provided by Genome System Corp.) where the D17S926 locus exists is obtained by shot gun method (made by Genecom, Inc.). Computerized recognition and prediction of new gene in PAC579 genomic sequence are carried out by using Bioinformation system (Celera) and Software “Undergo” (Axys Pharmaceuticals). The results show that there is a new gene. The position of this gene in PAC579 and the predicted exons are listed in the following table.

| symbol | number of exon (bp) | position in PAC579 | chain |
|---|---|---|---|
| | exon 1(122) | 50808-50687 | |
| CT120 | exon 2(169) | 45607-45406 | — |
| | exon 3(123) | 42939-42817 | |
| | exon 4(599) | 42143-41545 | |

### EXAMPLE 2

### Cloning of Full-Length cDNA of New Gene CT120

The human EST database is inquired by using the predicted exon sequence. A cDNA sequence FLJ22282 (GenBank No. AK025935) is obtained by assembling the returned EST sequences. Primers are designed based on this sequence for RACE reaction.

2.1 Reagents: cDNA pools (Human kidney Marathon-Ready cDNAs, Clontech), polymerase system (Advantage cDNA polymerase Mix, Clontech) TA cloning system (TOPO TA cloning).

2.2 Primer designing: The specific primers useful in RACE (Rapid amplification of cDNA ends) reaction should meet the following requirements: (a) the length should be within 23-28 nt; (b) GC content should be within 50-70%; (c) Tm value should be greater than 65° C. The following gene specific primers are designed and synthesized.

| | | | | |
|---|---|---|---|---|
| 120G | R | 5′GTGCGACTGGCACAAGGACAAAGAG3′ | (SEQ ID NO:3) | 5′ RACE |
| 120QNG | R | 5′CGAATGATGACGATCCCCGAGCC3′ | (SEQ ID NO:4) | 5′ RACE |

2.3 RACE reaction: PCR amplification may be carried out in the reaction volume of 12.5 μl or 25 μl. The conditions of RACE reaction are as follows:

| | total volume 12.5 μl |
|---|---|
| Marathon-Ready cDNAs | 1.25 μl |
| adapter primer | 0.25 μl |
| 10 mM dNTP | 0.25 μl |
| 10 × PCR reaction buffer | 1.25 μl |
| 50 × polymerase system | 0.25 μl |
| H₂O | 9.0 μl |
| gene specific primer (10 pmol/μl) | 0.25 μl |

Condition of PCR Reaction:

| | | |
|---|---|---|
| 94° C. | 1 min | 1 cycle |
| 94° C. | 30 sec | 5 cycle |
| 72° C. | 4 min | |
| 94° C. | 30 sec | 5 cycle |
| 70° C. | 4 min | |
| 94° C. | 20 sec | 25 cycle |
| 68° C. | 4 min | |

Subcloning of RACE product: The recovered PCR product (0.5-2.5 μl) and PCR-TOPO vector (Clontech) (0.5 μl) are mixed and put under room temperature for 5 minutes. Then, they are put on ice and transformed into bacteria by conventional methods. The coated plates are grown for 12-16 hours under 37° C. for white and blue spot screening.

2.4 Screening and Identifying the RACE Product

30 μl of LB of Amp resistance are added into each wells of 96-well plate. For each RACE reaction, 10-20 recombinant in white spots are picked onto the LB of the above 96-well plate. PCR reaction is directly carried out with said bacterial solution as a template. Positive RACE clone candidates are initially screened out, a small amount of which are subjected to liquid amplification. The plasmid DNA is extracted and treated by endonuclease. After electrophoresis analysis, a large segment of RACE clone is screened out for further PCR identification.

2.5 Sequencing of RACE Product and Analysis of Sequence:

The large segment of positive clone candidate is sequenced. It is determined whether the full length of said gene has been obtained, based on the length of RACE product and the length of mRNA of the gene. The full length sequence includes the complete reading-frame. There is a termination codon in frame before the first initiator codon ATG. There is a poly A sequence at the 3′ end of the reading-frame. Additionally, corresponding 5′ and 3′ non-encoding regions are included. The sequence and corresponding encoding frame of CT120, obtained by RACE method, are shown in SEQ ID NO: 1-2.

Among them, the full-length cDNA of CT120 has 2145 bps (SEQ ID NO: 1). The ORF comprises positions 91-861 which encodes a full length protein of 257 amino acids (SEQ ID NO: 2).

2.6 Homology Comparison

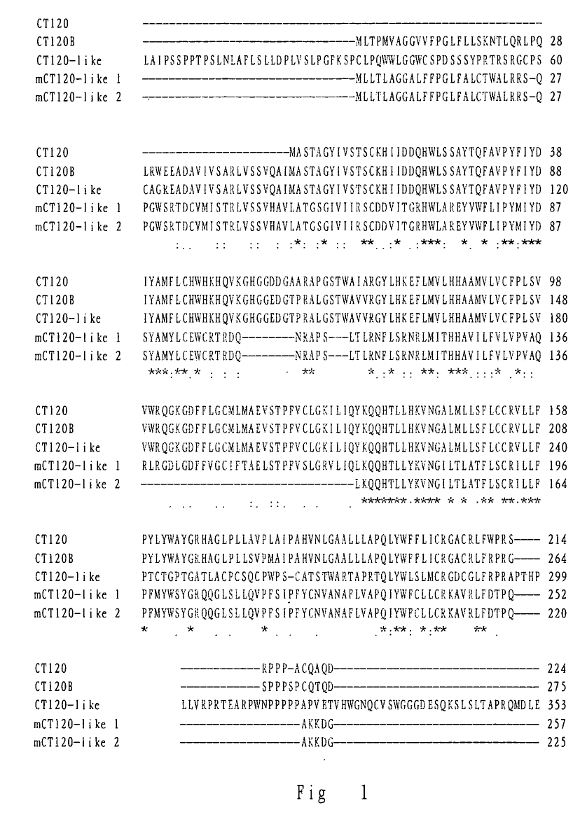

The result of homology comparison shows that the homologue of CT120 exists in different species. CT120 has two isoforms in human, one is the protein CT120A in the present invention, and the other is T120B (AAH26023). Compared with CT12A, CT120B lacks the fourth exon (96 bps, 32 amino acids). There is another CT120-like gene in human (NP-113666.1). There are two cognates, XP-133706 (named as “mCT120-like 1”) and BAB23923 (named as “mCT120-like 2”) in murine. The homology comparison is also shown in FIG. 1, wherein CT120 and CT120B have 223/257 (86%) of identity, CT120 and CT120-like have 104/210 (49%) of identity, CT120 and mCT120-like 1 have 126/260 (48%) of identity, and CT120 and mCT120-like 2 have 98/228 (42%) of identity.

2.7 Structural Analysis of CT120

Structural analysis is carried out on the nucleotide sequence and amino acid sequence of CT120. It is found that the CT120 polypeptide comprises the following potential functional domains and has seven transmembrane regions.

| name | position in SEQ ID NO: 2 |
|---|---|
| phosphorylation site of PKC | 39, 67, 109, 190 |
| phosphorylation site of Casein kinase II | 31, 61 |
| site of N-myristovl | 148 |
| cell adhesion sequence | 139-141 |
| signal peptide | 1-18 |
| transmembrane region I | 4-23 |
| transmembrane region 2 | 42-61 |
| transmembrane region 3 | 76-93 |
| transmembrane region 4 | 113-135 |
| transmembrane region 5 | 145-167 |
| transmembrane region 6 | 179-201 |
| transmembrane region 7 | 216-238 |

2.6 Full-Length Cloning of CT120:
Primers are designed based on the full-length sequence obtained from RACE reaction and then full-length cloning is carried out. The used primers are as follows:
120F1 F: 5′CCGATGCTGCTGACGCTGGCCG3′ (SEQ ID NO: 5)
120ER: 5′TGTTGGCACCAGAAAATCCTGCTTG3′ (SEQ ID NO: 6)

The condition of amplification employs RACE 25 μl reaction system and PCR reaction condition. After amplification, the full-length sequence (1907 bp) of CT120 is obtained. Then, it is inserted into T-A vector (Clontech) to obtain the vector CT 120-T-A.

### EXAMPLE 3

### Northern Hybridization of Multiple Tissue Membrane of CT120

The human multiple tissue membrane for Northern hybridization (MTN) is obtained from Clontech. Pre-hybridization is performed for 3-4 hours at 42° C. The CT120-T-A clone is cleaved by EcoRI for recovering the inserting fragment. Then, it is quantified by electrophoresis. 25 ng DNA is taken and added into 2.5 μl random primers and proper amount of water to achieve a total volume of 13.5 μl. After boiling for 5 minutes, the liquid is centrifuged to the bottom of the tube. Then, 2.5 μl of reaction buffer, 1 μl of each of dATP, dTTP, dGTP, 1 μl of Klenow enzyme, 5 μl of 32P-α-dCTP are added therein. With gentle mixing and centrifuging, the system is incubated for 20 minutes under 37° C. 2 μl of 0.5M EDTA is then added for stopping the reaction. A glass wool is plugged in a 1 ml injector and then TE-saturated Sephadex G-50 is added therein. It is then centrifuged at 200 rpm for 5 minutes and repeated for once. G-50 is added to be close to the scale 1 ml. Then, it is equilibrated by using 100 μl TE for three times. 75 μl of TE is added into the labeled reaction. After passing a column, it is recovered by centrifugation. The probe is denatured at 100° C. for 5 minutes and then is put on ice. Then, it is added into pre-hybridizing solution for hybridization of 12-16 hours at 42° C. The membrane is taken out and washed in 1× SSC-0.05% SDS solution at 42° C. for twice, each for 30 minutes. Then, it is washed in 0.1×SSC-0.1% SDS solution at 42° C. for twice, each for 30 minutes. Finally, autoradiography is performed using X-ray film.

The result of Northern hybridization is shown in FIG. 2. The CT120 gene has a full length of about 2.3 kb. It is expressed in heart, brain, placenta, liver, kidney, pancreas, skeletal muscle, but not in lung.

### EXAMPLE 4

### Semi-Quantitative RT-PCR

This example shows the expression of CT120 in different tumor cell lines through RT-PCR detection. The used tumor cell lines include pulmonary cancer SPC-A-1, cervical carcinoma C-33A, hepatoma SMMC-7221, BEL-7402, oophoroma SK-OV-3, bladder cancer 5637, epidermoid carcinoma A431, and breast cancer MCF-7.

4.1 reverse transcription: The first chain cDNA is synthesized according to the protocol of Superscript II RT kit (GIBCO, BRL) by using 1 μl of tissue total RNA and the total reaction volume is 20. After synthesis, the reaction volume is diluted to be 120. 1 μl of solution contains about 8 ng of RNA. The first chain cDNA is obtained after reverse transcription.

4.2 PCR Reaction System:

The following agents are added sequentially:

| | | |
|---|---|---|
| the first chain cDNA in the reverse transcription | 1 | μl |
| 10 × PCR buffer | 1.5 | μl |
| 2 mM dNTP | 1.5 | μl |
| BA1 primer (upstream) | 1.5 | μl |
| BA2 primer (downstream) | 1.5 | μl |
| CT120 F (upstream) | 1.5 | μl |
| CT120 G (downstream) | 1.5 | μl |
| Taq enzyme (promega 0.5 u/ul) | 1 | μl |
| H₂O | X | μl |
| total volume | 25 | μl |

4.3 PCR reaction procedure: 94° C., 3 min; 94° C. 30 sec; 60° C. 30 sec; 72° C. 30 sec; 26-28 cycles; 72° C., 5 min. After PCR reaction, 5 μl PCR product is taken for analysis via 2% agarose gel electrophoresis.

| | | | | |
|---|---|---|---|---|
| β-actin | BA1 | F | 5′AAGTACTCCGTGTGGATCGG3′ | SEQ ID NO:7 |
| | BA2 | R | 5′TCAAGTTGGGGGACAAAAAG3′ | SEQ ID NO:8 |
| CT120 | 120G | R | 5′GTGCGACTGGCACAAGGACAAAGAG3′ | SEQ ID NO:9 |
| | 120F | F | 5′GGGGATCGTCATCATTCGCTCCT3′ | SEQ ID NO:10 |

4.3 Result

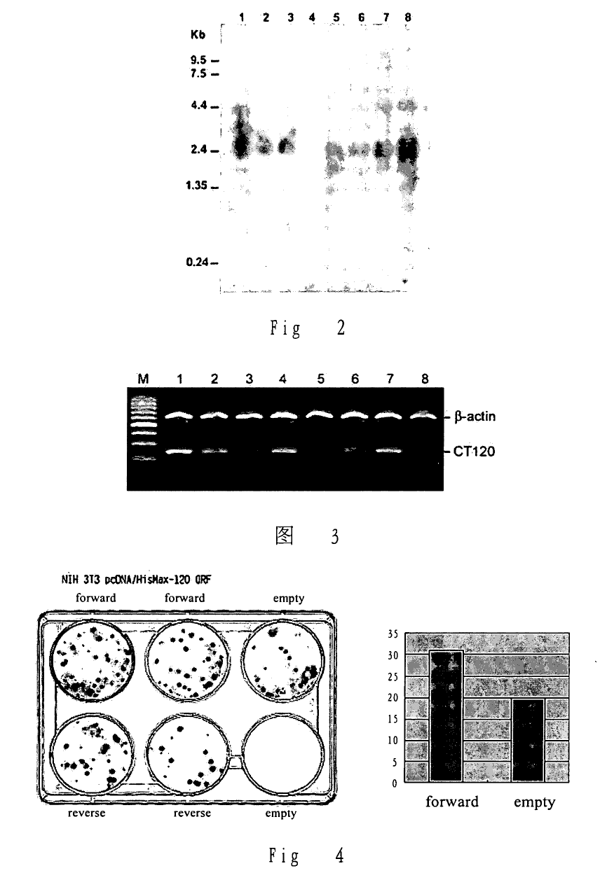

The result is shown in FIG. 3. CT120 exhibits high expression in pulmonary cancer cell line SPC-A-1, moderate expression in BEL-7402 and A431, and low expression in C-33A, SMMC-7721, 5637, MCF-7, and lower expression in SK-OV-3.

Since CT120 is not expressed in normal lung tissue but in pulmonary cancer cell, the detection of CT120 may be used to diagnose lung cancer.

### EXAMPLE 5

### Insertion of CT120 into Eucaryotic Expression Vector

pcDNA4/HisMax(Invitrogen) is selected to be the eucaryotic expression vector. ORF of CT120 is obtained through amplification by using 120HM-F: 5′ATGCTGCTGACGCTGGCCGG 3′ (SEQ ID NO: 12); 120HM-R: 5′TTAGCCATCCTTTTTGGCTT 3′ (SEQ ID NO: 13) as primers and cDNA pool (Clontech) as a template. T-A clone (Clontech) is cloned into pcDNA4/HisMax eucaryotic expression vector to obtain plasmid pcDNA4/HisMax-CT120 which is verified by sequencing. Clones are picked for amplification, extraction and cleavage, and then are used to transform the cells.

### EXAMPLE 6

### In Vitro Transfection of Cells by Using Liposome Transfection Kit

6.1 Cell strain: NIH/3T3 Cell

6.2 DNA: DNA from pcDNA4/HisMax-CT120 Expression Vector

6.3 Liposome: LIPOFECT AMINE™ Reagent Kit (BRL)

6.4 Culture Solution: Serum-Free Medium (SF-DMEM)
whole culture solution (10% fetal calf serum)
whole culture solution containing Zeocin (Invitrogen)
six-well plate (Corning)

6.5 Preparation of DNA-Liposome Complex:

10 μl of lipofectin is mixed thoroughly with 90 μl of SF-DMEM. 1 μg of DNA is added into 100 μl of SF-DMEM and mixed to homogeneity. The diluted DNA is added into the diluted lipofectin solution, mixed to homogeneity and put under room temperature for 30-45 minutes. 0.8 ml of SF-DMEM is added into DNA-lipofectin complex to a final volume of 1.0 ml.

6.6 Cell Transfection:

The cells are preferably grown to 50-60% confluence. The culture medium is renewed once before the experiment. 1.0 ml of lipofectin Reagent-DNA complex is added onto the surface of cells, spread evenly by shaking gently, and then incubated under 37° C. for 5 hours. 1 ml of DMEM containing 20% fetal calf serum is then added and mixed. The cells are grown overnight under 37° C. The culture medium is renewed for overnight incubation, and then is replaced with whole culture solution containing Zeocin. Then, the culture medium is conventionally renewed for screening until clones appear. The number of the clones is recorded.

The result is shown in FIG. 4. CT120 obviously promotes the growth of NIH/3T3 cells.

6.7 Establishment of NIH/3T3 Cell Lines Steadily Transfected by CT120:

A single cell clone of NIH/3T3 transfected steadily by CT120 is picked for amplification. 12% SDS-PAGE electrophoresis is carried out for the lysate of the clone and transferred to the membrane. Anti His G (Invitrongen) labeled MAb is used to detect the expression of CT120 fusion protein in the steadily transfected NIH/3T3 cell line.

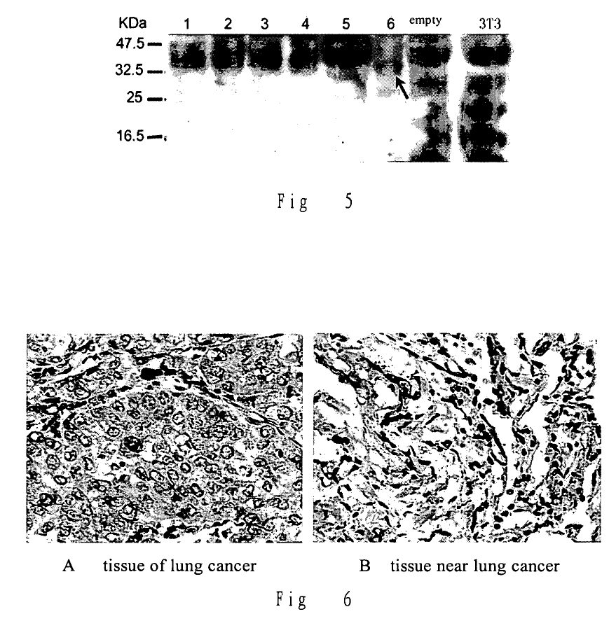

The result is shown in FIG. 5. Five clones among six tested clones show the expression of CT120. The molecular weight of CT120 is about 34 KDa.

### EXAMPLE 7

### Detection of Expression of CT120 in Tissue of Pulmonary Cancer and the Tissue Surrounding the Pulmonary Cancer by Immunohistochemistry Detection

7.1 Preparation of Rabbit Anti-CT120 Polyclonal Antibody:

Oligopeptide (15 amino acids) of C-terminus of CT120 protein (CRKAVRLFDTPQAKK (SEQ ID NO: 11)) is synthesized by peptide synthesizer (Applied Biosystem). The synthesized polypeptide is coupled to KLH by using Maleimide Activated BSA and KLH coupling kit (Sigma), and then is used to immunize the new zealand rabbit to prepare rabbit anti-CT120 polyclonal antibody.

7.2 Detection of Expression of CT120 in Tissue of Pulmonary Cancer and the Tissue Near the Pulmonary Cancer by Immunohistochemistry Detection

The tissue of pulmonary cancer and the tissue near the pulmonary cancer are taken from the clinically excised tissues of pulmonary cancer patients, fixed by 10% neutral formalin buffer for immunohistochemistry detection, embedded in paraffin wax and sliced into 5 μm slice. The rabbit anti-CT120 polyclonal antibody (1:150 dilution) is used as the first antibody. Envision System two-steps method detection kit (mouse) is used. It is developed by DAB and Mayer's haematoxylin is used for staining the nucleus.

The result is shown in FIG. 6. CT120 gene is highly expressed (++) in the cancer cells in tissue of pulmonary cancer, while it is almost not expressed (−−) in the tissue near the pulmonary cancer.

All the documents cited herein are incorporated into the invention as reference, as if each of them is individually incorporated. Further, it would be appreciated that, in the above teaching of the present invention, the skilled in the art could make certain changes or modifications to the invention, and these equivalents would still be within the scope of the present invention defined by the appended claims of the application.

What is claimed is:

1. An isolated human CT120 protein polypeptide comprising the amino acid sequence of SEQ ID NO: 2, or its conservative variant polypeptide, active fragment or active derivative.

2. The polypeptide of claim 1, wherein said polypeptide is selected from the following group:
(a) a polypeptide having the amino acid sequence of SEQ ID NO: 2;
(b) a polypeptide derived from the polypeptide (a) with one or more substitution, deletion or insertion in the amino acid residues of polypeptide (a), which has the function of promoting the growth of NIH/3T3 cell.

3. An isolated polynucleotide comprising a nucleic acid sequence selected from the following group:
(a) polynucleotides encoding the polypeptide according to claims 1 and 2;
(b) polynucleotides which are complementary to polynucleotides (a).

4. The polynucleotide of claim 3, wherein the polypeptide encoded by said polynucleotide has the amino acid sequence of SEQ ID NO: 2, or said polynucleotide has a sequence selected from the following group: the sequence in encoding region of position 91-861 of SEQ ID NO: 1 or the fill length sequence position 1-2145 of SEQ ID NO: 1.

5. A vector comprising the polynucleotide of claim 3.

6. A genetic engineered host cell selected from the following group:
(a) host cell transformed or transduced by the vector of claim 5;
(b) host cell transformed or transdues by the polynucleotide of claim 3.

7. A method for producing a polypeptide comprising:
(a) culturing the host cell of claim 6 under the conditions suitable for the expression of protein;
(b) isolating the polypeptides having the activity of tumor-related protein CT120 from the culture.

8. An antibody specifically binding with the human CT120 of claim 1.

9. A method for detecting the carcinomatous change or susceptibility for carcinomatous change in pneumonocytes, comprising the steps of:
detecting whether there is any CT120 transcript in the pneumonocyte sample, the presence of CT120 transcript indicating that said pneumonocyte undergoes carcinomatous change or has the susceptibility for carcinomatous change; or
detecting whether there is CT120 protein in the pneumonocyte sample, the presence of CT120 protein indicating that said pneumonocyte undergoes carcinomatous change or has the susceptibility for carcinomatous change.

10. A kit for detecting pulmonary cancer, which comprises (1) primers which specifically amplify human CT120 gene, or (2) antibody specifically binding with CT120 protein.


SEQUENCE LISTING (13 sequences)

SEQ ID NO: 1 - DNA, 2145 bp, Homo sapiens
  CDS (91)..(861)
cggagggttgaaatcgcgcggccgggccggggcgcgccgagccgaacccagccacgcggcgccagcgaggcggccggacccgcagccccgatgctgctgacgctggccgggggcgcgctcttcttcccggggctcttcgcgctctgcacctgggcgctgcgccactcccagcccggatggagccgcaccgactgcgtgatgatcagcaccaggctggtttcctcggtgcacgccgtgctggccaccggctcggggatcgtcatcattcgctcctgcgacgacgtgatcaccggcaggcactggcttgcccgggaatatgtgtggtttctgattccatacatgatctatgactcgtacgccatgtacctctgtgaatggtgccgaaccagagaccagaaccgtgcgccctccctcactcttcgaaacttcctaagtcgaaaccgcctcatgatcacacatcatgcggtcattctccttgtccttgtgccagtcgcacagaggctccggggagaccttggggacttctttgtcggctgcatcttcacggcagaactgagcactccgtttgtgtcgctgggcagggttctgattcagctaaagcagcagcacacccttctgtacaaggtgaatggaatcctcacgctggccaccttcctttcctgccggatccttctcttccccttcatgtactggtcctatggccgccagcagggactaagcctgctccaagtacccttcagcatcccattctactgcaacgtggccaatgccttcctcgtagctcctcagatctactggttctgtctgctgtgcaggaaggcagtccggctctttgacactccccaagccaaaaaggatggctaaatgctcctgggagtcaggcgcagcctcacaccagctgcctcctccactcagcattccatggaccaaattgtgccctgggtagcctcagactttgggtattgataagccgatggatttgagtttttctaaagaatattcatattacctcctttttctaacttgccctatttgcaaacgcacttttgtagtaacaactattgggtcctgtcagacctccacggacagcaaagtggttttaatgcaagcccaaggatccttcttaaggtcttatctcaagagctctgggaggtggaagcatggggtgggatcggtggaccagggtggtaagtgtctgcacatctgcctgtccctgtatcagcggctacccaccttccaaaccactcaggacagtacccgtggcactgggcccgcagaagcaagggatgacttggttcttggaagtaatgtcgtcttgtgacattggcctgggacaatcattgtgggtaggtagttattgatcgtttactagataacccattggttctttgcctcatcctctcatccatgggtcagagttgaattcttatgtctatagacttccaatcagaagtctcactggtggggctgggggtgggggcaggcaggaggcatggatgggaacctgagtaggtagtgtggccaagagatcagcacaacctttgcaggctgacttgctaagtctgacagtgacaaacttgtgagcttactgcagtcagtcacagaggctgttctttttcacacaccccttcatgcccggctttccccatatccacatgcagagggcgagctcataaaactacagggaagcgtgaaatgatggctttggtagctgtttactgggtaaccccactgtgacactgtccttttcatgtgatgtggaaacctacttctgtcctccaaaccatgaaatgtgtcatctagactgcagagtactcgagtgctttgcctcccgatatgccagagcttgtggtccaaagcccattcctgtgtgtccgtcctgccatttagccacagaaggctgcggagtgaggcggcagctagcctggccagtggctgtcccgtggaccgacacctgcgcccccttctgcaagcaggattttctggtgccaacactcattcatcattcccgatcaactaggatgaatttaagactgtgctaccatgtgttctcaagtggtagtttaaaaagtggatttttaaagtgcctttcaattgtctgtgaacgtctaaaggactgatttgtctcaaaaaaaaaaaaaaaaa

SEQ ID NO: 2 - protein, 257 aa, Homo sapiens [sequence omitted]

SEQ ID NO: 3 - DNA, 25 bp, Artificial
  misc_feature (1)..(25): primer
gtgcgactggcacaaggacaaagag

SEQ ID NO: 4 - DNA, 23 bp, Artificial
  misc_feature (1)..(23): primer
cgaatgatgacgatccccgagcc

SEQ ID NO: 5 - DNA, 22 bp, Artificial
  misc_feature (1)..(22): primer
ccgatgctgctgacgctggccg

SEQ ID NO: 6 - DNA, 25 bp, Artificial
  misc_feature (1)..(25): primer
tgttggcaccagaaaatcctgcttg

SEQ ID NO: 7 - DNA, 20 bp, Artificial
  misc_feature (1)..(20): primer
aagtactccgtgtggatcgg

SEQ ID NO: 8 - DNA, 20 bp, Artificial
  misc_feature (1)..(20): primer
tcaagttgggggacaaaaag

SEQ ID NO: 9 - DNA, 25 bp, Artificial
  misc_feature (1)..(25): primer
gtgcgactggcacaaggacaaagag

SEQ ID NO: 10 - DNA, 23 bp, Artificial
  misc_feature (1)..(23): primer
ggggatcgtcatcattcgctcct

SEQ ID NO: 11 - protein, 15 aa, Artificial
  MISC_FEATURE (1)..(15): oligopeptide corresponding to C-terminus of CT120 protein
CRKAVRLFDTPQAKK

SEQ ID NO: 12 - DNA, 20 bp, Artificial
  misc_feature (1)..(20): primer
atgctgctgacgctggccgg

SEQ ID NO: 13 - DNA, 20 bp, Artificial
  misc_feature (1)..(20): primer
ttagccatcctttttggctt